SEQUENCE LISTING (56 sequences)

SEQ ID NO: 1 - DNA, 28 bp, Borrelia burgdorferi
gattcaaataaaaattctaaattaccat

SEQ ID NO: 2 - DNA, 22 bp, Chlamydia pneumoniae
agttacctggactcaaataaaa

SEQ ID NO: 3 - DNA, 14 bp, Borrelia burgdorferi
gtttaataaaaatt

SEQ ID NO: 4 - DNA, 15 bp, Chlamydia pneumoniae
agtttaataaagatt

SEQ ID NO: 5 - DNA, 16 bp, Borrelia burgdorferi
atactaatatcaataa

SEQ ID NO: 6 - DNA, 18 bp, Plasmodium falciparum
ttttcatgtaaataaata

SEQ ID NO: 7 - DNA, 17 bp, Borrelia burgdorferi
tttaaataaaaataagt

SEQ ID NO: 8 - DNA, 25 bp, Plasmodium falciparum
gattttttctttagataaaaataag

SEQ ID NO: 9 - DNA, 16 bp, Borrelia burgdorferi
acaataaataatattc

SEQ ID NO: 10 - DNA, 16 bp, Borrelia burgdorferi
taataataaaaatcat

SEQ ID NO: 11 - DNA, 19 bp, Escherichia coli
aataataaaaatcatgctt

SEQ ID NO: 12 - DNA, 16 bp, Plasmodium falciparum
aataataaaaatcatg

SEQ ID NO: 13 - DNA, 18 bp, Borrelia burgdorferi
tccggaataaaaggccct

SEQ ID NO: 14 - DNA, 16 bp, Chlamydia muridarum
gcgaataaaaggccct

SEQ ID NO: 15 - DNA, 16 bp, Rickettsia prowazekii
aataaaaaacatttat

SEQ ID NO: 16 - DNA, 20 bp, Borrelia burgdorferi
aaactcagaaaataaaatgt

SEQ ID NO: 17 - DNA, 20 bp, Borrelia burgdorferi
caaaataaagtagtaaaaga

SEQ ID NO: 18 - DNA, 24 bp, Borrelia burgdorferi
catttcttcaactaaattaacatt

SEQ ID NO: 19 - DNA, 27 bp, Homo sapiens
attcatttcttcaactaaattaacatt

SEQ ID NO: 20 - DNA, 22 bp, Chlamydia sp.
ttacctggactcaaataaaagt

SEQ ID NO: 21 - DNA, 36 bp, Rattus norvegicus
ttacctggaatcaaataaaagttctagattatcacg

SEQ ID NO: 22 - DNA, 38 bp, ancestor rodent
gaatcaaataaaaattctagattaccatgaagaacata

SEQ ID NO: 23 - DNA, 17 bp, Borrelia garinii
ataataattctaaatta

SEQ ID NO: 24 - DNA, 17 bp, Borrelia burgdorferi
ataataattctaaatta

SEQ ID NO: 25 - DNA, 16 bp, Borrelia sp.
aatataaattctatat

SEQ ID NO: 26 - DNA, 14 bp, ancestor human
gtttaataaaaatt

SEQ ID NO: 27 - DNA, 17 bp, Homo sapiens
agtttaataaagattca

SEQ ID NO: 28 - DNA, 15 bp, Borrelia sp.
tttaaaaaagattca

SEQ ID NO: 29 - DNA, 17 bp, Homo sapiens
atactaatatcaataaa

SEQ ID NO: 30 - DNA, 17 bp, Borrelia burgdorferi
atactaatatcaataat

SEQ ID NO: 31 - DNA, 24 bp, Homo sapiens
ttttcatgtaaataaatacattct

SEQ ID NO: 32 - DNA, 17 bp, Plasmodium falciparum
tttcaggtaaataaata

SEQ ID NO: 33 - DNA, 26 bp, Homo sapiens
gattttttctttaaataaaaataagt

SEQ ID NO: 34 - DNA, 21 bp, Plasmodium falciparum
ttttttttttaaataaaaata

SEQ ID NO: 35 - DNA, 21 bp, Homo sapiens
cctaataaaggaatagttaac

SEQ ID NO: 36 - DNA, 16 bp, Homo sapiens
gctagtaataaaatat

SEQ ID NO: 37 - DNA, 16 bp, Homo sapiens
acaataaataatattc

SEQ ID NO: 38 - DNA, 18 bp, Homo sapiens
tccggaataaaaggccct

SEQ ID NO: 39 - DNA, 18 bp, Homo sapiens
tcgcgaataaaaggccct

SEQ ID NO: 40 - DNA, 43 bp, Homo sapiens
aataaaaaacatttattttcattgcaatgatgtatttaaatta

SEQ ID NO: 41 - DNA, 12 bp, Borrelia sp.
atgtatttaaat

SEQ ID NO: 42 - DNA, 22 bp, Homo sapiens
tttctgaatattttactaaaaa

SEQ ID NO: 43 - DNA, 21 bp, Borrelia sp.
ttctgaagattttactaaaaa

SEQ ID NO: 44 - DNA, 26 bp, Homo sapiens
aataagaaacatttattttcattgca

SEQ ID NO: 45 - DNA, 17 bp, Plasmodium falciparum
ataagaaacatttattt

SEQ ID NO: 46 - DNA, 576 bp, Homo sapiens
actcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgccctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtggccatgcttcttgccccttgggcctccccccagcccctcctccccttcctgcacccgtacccccgtggtctttgaataaagtctgagtgggcggc

SEQ ID NO: 47 - DNA, 47 bp, Homo sapiens
agttacctggaatcaaataaaaattctagattaccatgaagaacata

SEQ ID NO: 48 - DNA, 18 bp, Salmonella typhimurium
tgtaaataaatacattct

SEQ ID NO: 49 - DNA, 20 bp, Homo sapiens
taataataaaaatcatgctt

SEQ ID NO: 50 - DNA, 14 bp, ancestor human
gtttaataaaaatt

SEQ ID NO: 51 - DNA, 16 bp, Borrelia sp.
tttaaataaaaataag

SEQ ID NO: 52 - DNA, 17 bp, Vibrio cholerae
aataaaggaatagttaa

SEQ ID NO: 53 - DNA, 16 bp, Clostridium sp.
cctaataaaggaatag

SEQ ID NO: 54 - DNA, 20 bp, Homo sapiens
aaactcagaaaataaaatgt

SEQ ID NO: 55 - DNA, 12 bp, Borrelia burgdorferi
cagaagtaaaat

SEQ ID NO: 56 - DNA, 19 bp, Homo sapiens
caaataaagtagtaaaaga

(19) United States
(12) Patent Application Publication
(10) Pub. No.: US 2004/0101867 A1
(43) Pub. Date: May 27, 2004

(54) Use of microbial dna sequences for the identification of human diseases

(76) Inventor: Markus Fritzsche, Adliswil (CH)

Correspondence Address: KENYON & KENYON, 1500 K STREET, N.W., SUITE 700, WASHINGTON, DC, 20005, US

(21) Appl. No.: 10/344,124

(22) PCT Filed: Feb. 15, 2001
(86) PCT No.: PCT/IB01/00189

(51) Int. Cl. C12Q 1/68; C07H 21/04

(52) U.S. Cl. 435/6; 536/23.7

(57) ABSTRACT

The use of DNA sequences comprising a fragment of a nucleic acid encoding a microbial virulence factor as means for the identification of diseases or a genetic predisposition thereof as well as its use for the development of disease animal models is disclosed.

### CROSS REFERENCE TO RELATED APPLICATION

This application claims the priority of PCT patent application IB00/01127, filed Aug. 16, 2000, the disclosure of which is incorporated herein by reference in its entirety.

### TECHNICAL FIELD

The present invention relates to the use of a DNA sequence comprising a fragment of a nucleic acid encoding a microbial virulence factor as means for the identification of a disease or a genetic predisposition thereof as well as its use for the development of disease animal models.

### BACKGROUND ART

The functional sequences of higher eukaryotes consist of genetic modules of at least two kinds. Modules of coding sequence are combined in many ways to produce proteins, whereas modules of non-coding sequences regulate the expression of genes. Some of the mutations created duplicates of entire genes, which have then evolved new functions, while others altered the expression of old genes by exposing them through gene shuffling to new regulatory sequences. By these means the human genome as a whole has evolved to its present day complexity. Since the 3′ untranslated region (3′UTR) and especially the polyadenylation signal within the 3′ UTR regulate the translation and expression of a gene, the entire gamut of molecular perturbations can be accomplished with the 3′UTR as a primary target.

There is therefore a need for molecular tools allowing the detection of diseases or a predisposition thereof caused by mutations within the 3′ UTR.

### DISCLOSURE OF THE INVENTION

In the scope of the present invention it was now found that there are microbial DNA insertions in the 3′ UTR of human genes which are associated with human diseases.

Hence it is an object of the present invention to provide the use of DNA sequences comprising a fragment of a nucleic acid sequence encoding a putative microbial virulence factor as means for the identification of diseases caused by bacterial mutations or a genetic predisposition thereof. The virulence factor stems preferably from a intracellular microorgansim and is located on a linear or circular chromosome or a plasmid, more preferably said virulence factor stems from a microorganism which is selected from the group consisting of Borrelia species, Chlamydia sp., Escherichia sp., Plasmodium sp. and Rickettsia. Even more preferably said nucleic acid encoding a virulence factor is selected from the group consisting of Seq. Id. No. 1 to Seq. Id. No. 17. Virulence factors stemmming from non-intracellular microorganisms which are part of a cluster shared by intracellular microonganisms are as well suitable for the use in the present invention.

Another object of the present invention is a method for the identification of a disease or a genetic predisposition thereof, which comprises in a tissue or blood sample of a subject or in a fetal neuro-graft a mutation within a nucleic acid sequence selected from the group consisting of Seq. Id. No. 1 to Seq. Id. No. 17 and said sequence is inserted in a gene of said subject.

Preferably said sequence is inserted in the 3′UTR of said gene and said mutation is found in the polyadenylation signal of said gene and said mutation preferably affects the expression of the protein encoded by said gene.

Another object of the present invention are transgenic non-human animals, which comprise in their genome a partial or complete inactive endogenous gene which is selected from the group consisting of cannabinoid receptor 1 gene, MAP 2C gene, apolipoprotein E gene, presenelin 2 gene, integral membrane protein 2B gene, alpha synuclein gene, oligophrenin 1 gene and myotonin protein kinase gene. The gene is inactivated due to at least one mutation in its 3′ untranslated region (3′ UTR) and said mutation leads to an inhibition or suppression of protein expression. The term mutation as used herein encompasses any nucleotide change, insertion or deletion independent of their length that influences the activity, expression or regulation of a gene.

Although in the context of the present invention any mutation in the 3′UTR region leading to an inhibition or suppression of protein expression e.g. CB1 protein expression, can be used, preferred are mutations located in the sequence following the polyadenylation signal, more preferably in the polyadenylation signal sequence of said gene. Any mutation in the polyadenylation signal sequence leading to an inactivation of said signal can be used. The mutation can e.g. be caused by a sequence of the same or a different microbial species e.g. by gene conversion or recombination.

The polyadenylation signal in eukaryotes has the following conserved sequence: AATAAA.

For the purpose of the present invention any non-human mammal can be used. Preferred are rodents e.g. mice or rats, which preferably harbor a homozygous or heterozygous CB1 gene inactivation in their genome.

Another object of the present invention is the use of the transgenic animals of the present invention for the identification of compounds that have an effect on the activity, expression or regulation of the gene encoded protein. The animals of the present invention allow the identification of compounds which have a direct or indirect effect on CB1 protein, MAP 2C protein, Apolipoprotein E, presenilin 2 protein, integral membrane protein 2B, alpha synuclein protein, oligophrenin 1 protein and myotonin protein kinase activity, expression or regulation. The compound may consist of a multiplicity of compounds e.g. as obtained from combinatorial chemical libraries. A compound identified by the use of the animals of the present invention can serve as a lead compound for the development of medicaments to treat e.g. schizophrenia or other forms of dementia.

A further object of the present invention is a method for screening for compounds that have an effect on the activity, expression or regulation of proteins selected form the group consisting of CB1 protein, MAP 2C protein, Apolipoprotein E, presenilin 2 protein, integral membrane protein 2B, alpha synuclein protein, oligophrenin 1 protein and myotonin protein kinase. Said method comprises introducing a compound in an transgenic animal of the present invention, preferably in a mouse or a rat, and monitoring e.g. behavioural changes in said animal. Mice and rats are preferred animals as their observable behavioural changes are considered relevant to clinical phenomenology. A transgenic animal, which harbors one of the above mentioned genes in its genome that is inactivated by a mutation in the sequence following the polyadenylation signal or in the polyadenylation signal is preferably used in a screening method of the present invention since such an animal reflects the situation found in human subjects suffering e.g. from schizophrenia, other forms of dementias and other neurological disorders.

In a further aspect the invention provides a use of a transgenic non-human animal whose genome comprises a disruption of the endogenous CB1 gene for the identification of compounds that have an effect on the activity, expression or regulation of CB1 protein. Any animal comprising a non-functional CB1 gene in its genome is suitable for such a use, preferably a mouse or a rat.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1 shows genetic exposure triggering gene conversion and further infectious recombination,

FIG. 2a shows genetic exposure leading to multiple translocations into the human genome and

FIG. 2b shows genetic exposure leading to multiple translocations into the human genome

### MODES FOR CARRYING OUT THE INVENTION

A gene construct for the production of a transgenic animal of the present invention, which comprises in its genome a partially or completely inactivated gene, can be prepared using standard genetic engineering technologies known in the art, such as described in Maniatis et al., Molecular cloning: A Laboratory Manual, Cold Spring Laboratory, Cold Springs Harbor, N.Y. The starting material for said construct can be a portion of e.g. the genomic or cDNA CB1 nucleotide sequence. Introduction of the wanted mutation in e.g. the CB1 sequence can be done by methods known to a person skilled in the art e.g. by site directed mutagenesis. The term mutation as used herein encompasses any nucleotide change, insertion or deletion independent of their length that influence the activity, expression or regulation of a gene.

A transgenic animal in accordance with the present invention can be made using generally known methods in the field. See, for example, Manipulating the Mouse Embryo, (Cold Spring Harbor Laboratory Press, Cold Spring Harbor, N.Y., 1986), Knockout mouse models used to study neurobiological systems, Critical Reviews in Neurobiology, 13 (2), 103-149, (1999). Recombinase dependent knockouts can also be generated, e.g. by homologous recombination to insert recombinase target sequences flanking portions of an endogenous gene, such that tissue specific and/or temporal control of inactivation of an allele can be controlled.

The transgenic animals of the present invenion are suitable to identify lead compounds that can serve as a starting point for the development of a medicament to treat e.g. schizophrenia, Alzheimer disease, Parkinson disease, Myopathy or other forms of dementia. Screening for such useful compounds typically involves administering the candidate compound to an animal and monitoring behavioural changes in said animal. Said behavioural changes include for example changes in locomotor activity, stereotypical behaviour, enhanced spatial memory and disruption of working memory. Also biochemical or molecularbiological assays can be used in a screening test for the identification of active compounds. Such tests include for example looking for increased or decreased expression and/or stability and/or activity of CB1 protein or increased or decreased levels and/or activity of messengers such as dynorphin, substance P in various tissues of the animals at different time points. At the end of the test the animals are preferably sacrificed to determine for example the effect of the administerd test compound on the expression level and/or expression sites of the CB1 protein in the brain by for example in situ techniques.

There are at least three different categories of compounds that can be screened by a screening test of the present invention: chemical libraries, natural product libraries and combinatorial libraries. Chemical libries consist of structural analogs of known compounds. Natural product libraries are collections of microorganism, animals, plants or marine organisms which are used to create mixtures for screening by for example fermentation and extraction of broths from soil, plant or marine microorganisms or extraction of plants or marine organisms. Combinatorial libraries are composed of large numbers of peptides, oligonucleotides or organic compounds as a mixture. They are relatively easy to prepare by traditional synthesis methods, PCR or cloning.

In the above described screening systems not only transgenic animals of the present invention but any transgenic animal comprising a non-functional gene encoding a protein selected from the group consisting of CB1 protein, MAP 2C protein, Apolipoprotein E, presenilin 2 protein, integral membrane protein 2B, alpha synuclein protein, oligophrenin 1 protein and myotonin protein kinase in its genome can be used. The non-functional gene can preferably be produced by introduction of at least one mutation in the coding-sequence and/or 5′ or 3′regulatory sequences or in an intron of the gene.

Microbial DNA sequence insertions found in several genes can be used to identify prenatal infectious diseases, bacterial mutations and other diseases, preferably human diseases, e.g. dementias or a predisposition thereof. Genes with such insertions are e.g. genes encoding the cannabinoid receptor, the microtubule associated protein 2C, apolipoprotein B, presenilin 2, amyloid precursor protein, integral membrane protein 2B, notch 3, microtubule associated protein tau, alpha synuclein and myotonin protein kinase. Said genes are known to be associated with the development or a predisposition for diseases like schizophrenia, Parkinson disease, Alzheimer disease, myopathy, frontotemporal lobe dementia, hereditary multi-infarct dementia, autosomal dominant Parkinson Lewy-Body dementia, familial British dementia and primary X-linked mental retardation. Said microbial DNA sequences are as well suitable for the detection of a predisposition or a genetic variation for diseases, the pathological manifestation of which is triggerd by medicaments or drugs like e.g. cannabis. For the above mentioned use sequences encoding microbial virulence factors and harboring at least one mutation affecting expression, activity or regulation of proteins encoded by said genes are preferred.

As already mentioned above the present invention also concerns methods for the identification of a disease caused by bacterial mutations or a genetic predisposition thereof. Said methods comprise detecting the presence in a tissue-or blood sample of a subject a mutation within a nucleic acid sequence selected from the group consisting of Seq. Id. No.1 to Seq. Id. No. 17 and said sequence is part of a gene of said subject. The method of the present invention is e.g. suitable for the identification of one of the above mentioned diseases.

Suitable methods for the identification of said diseases or a genetic predisposition thereof in humans are for example PCR techniques, DNA or gene chips, hybridisation techniques and Ligases chain reaction (LCR).

In a preferred embodiment said method comprises the following steps:

1. Blood or Tissue sample

2. DNA extraction

3. Amplification of at least one Sequence selected from the group consisting of Seq. Id. No. 1 to 17 using flanking oligonucleotide primers

4. Analysis of amplification products by sequencing

The sequencing step allows the identification of mutations present in the insertion sequence of interest. The design of the oligonucleotide primers is known to those skilled in the art and can be done using standard software.

### EXAMPLES

### Example 1

Microbial sequences in 3′UTR polyadenylation regions of schizophrenia and dementia genes harbour microbial virulence factors and plasmids

DNA from Borrelia burgdorferi, Chlamydia and other intracellular microbes has inserted into ancestral 3′ polyadenylation sites of the following candidate neurological genes: The central cannabinoid receptor gene (CB1), Alzheimer disease and other dementia genes, Parkinson disease and myotonic dystrophy (see Seq. Id. No. 1 to 17 and Table 1A to 1E). Most insertions originate from microbial virulence factors, transposable elements and plasmids.

CB1 and 5HT1E contain related insertions from B. burgdorferi. The human CB1 gene is located at 6q14, which has been reported as a candidate region for schizophrenia involving a translocation break-point co-segregating with schizophrenia, immediately adjacent to the 5-hydroxytryptamine (5HT1E) gene. The spirochaetal insertion into 5HT1E originated from a B. burgdorferi virulence factor, the flagellar basal-body rod protein (fbrp), responsible for chemotaxis, locomotion and a syringe mechanism for injection into cells. Through infectious recombination, B. burgdorferi introduced another nucleotide sequence containing p115 and a polyadenylation site into our ancestral genome. As a result of this recombination, p115 overlaps with fbrp. The fbrp section on the CB1 gene originates from an ancient 5HT1E receptor already containing the spirochaetal insertion and not from a direct transposition of B. burgdorferi onto 6q14, because the first three nucleotides of the overlapping sequence (see Table 1A) are on 5HT1E, and not on fbrp of B. burgdorferi. Gene conversion is the most likely explanation since CB1 and human 5HT1E, on which fbrp is absent, are both located in tandem on 6q14.

The spirochaetal recombination with p115 then led to the introduction of the poly-A signal (AATAAA) which codes for the translation and genetic expression of CB1. Being located on an important 3′ regulatory code, whose mutational rate runs slower than the molecular clock of silent point mutations in protein coding regions, microbial sequences are thus better protected from mutations and hence stabilised.

B. burgdorferi and Chlamydia pneumoniae have been detected in post mortem Alzheimer brains. Within the polyadenylation signal of the candidate gene apolipoprotein E (Apo E) epsilon 4 allele, which is a risk factor for schizophrenia, Alzheimer's disease, and chlamydial infection, the possible molecular basis for this—an overlapping sequence of microbial virulence factors originating from the linear chromosome of B. burgdorferi (including two copies on plasmids) and the human pathogenic species Chlamydia pneumoniae (see Table 1B) were identified.

The origin of new traits through lateral microbial gene transfer into mammals is still in its infancy and counter-current to the established views of understanding evolution through mutation and selection of existing sequences. Instead, multiple infectious recombinations and genetic exposure of pre-inserted templates has led to a further spread of Borrelia DNA all over the human genome. Intriguingly, by a single frame-shift mutation within the original translocase, genomic decay has apparently incapacitated B. burgdorferi to translocate further genetic templates into its hosts. Resulting from the same process of genomic decay in plasmids, or alternatively, from genetically induced variability by virulence factor operons, osp A and B, nucleotide dissimilarity between B. burgdorferi and its pre-inserted human templates have occurred. In the case of further infectious recombination with Osp A, this might trigger mismatch repair mutations within the poly-A signal and a genetic knock-out of CB1. The transition of the parasite from arthropod vector to human host is accompanied by significant changes in gene expression, which has practical relevance for prevention, as well. The recently approved vaccine against Lyme disease consists of an immunogenic Osp A, that is expressed by the spirochaetal parasite while in the tick gut, turned off on entry into humans and then expressed again at a late chronic stage. After attachment of an infected tick and initiation of a blood meal, anti-OspA antibodies enter the tick gut and mediate killing of B. burgdorferi.

### Example 2

Lateral Gene Transfer of B. burgdorferi into 5HT1E, Gene Conversion and Homologous Recombination with CB1

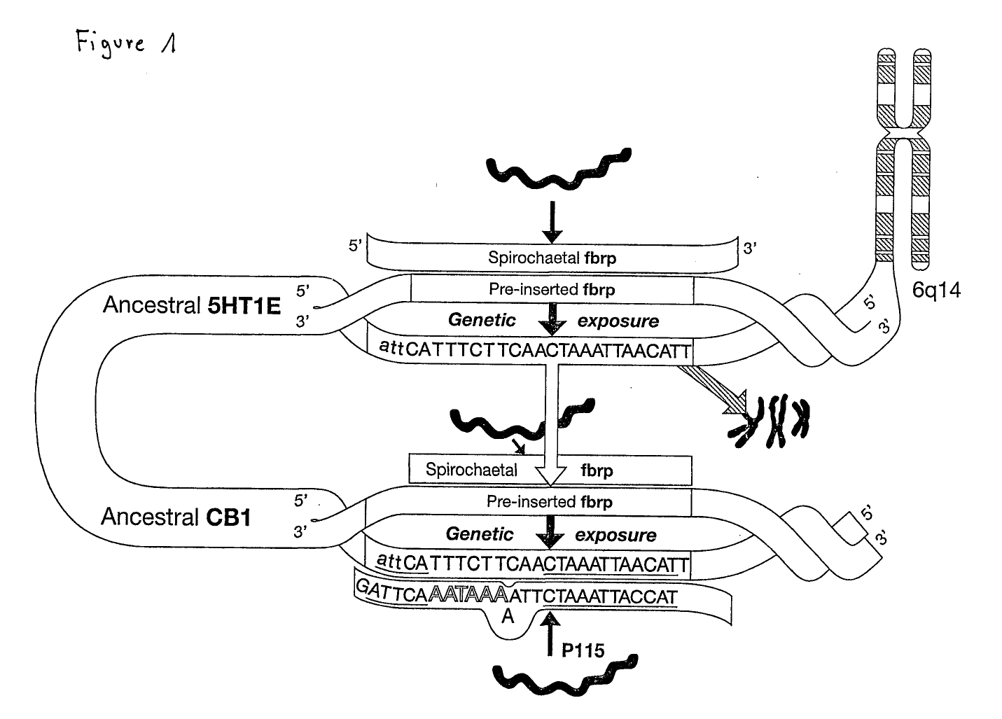

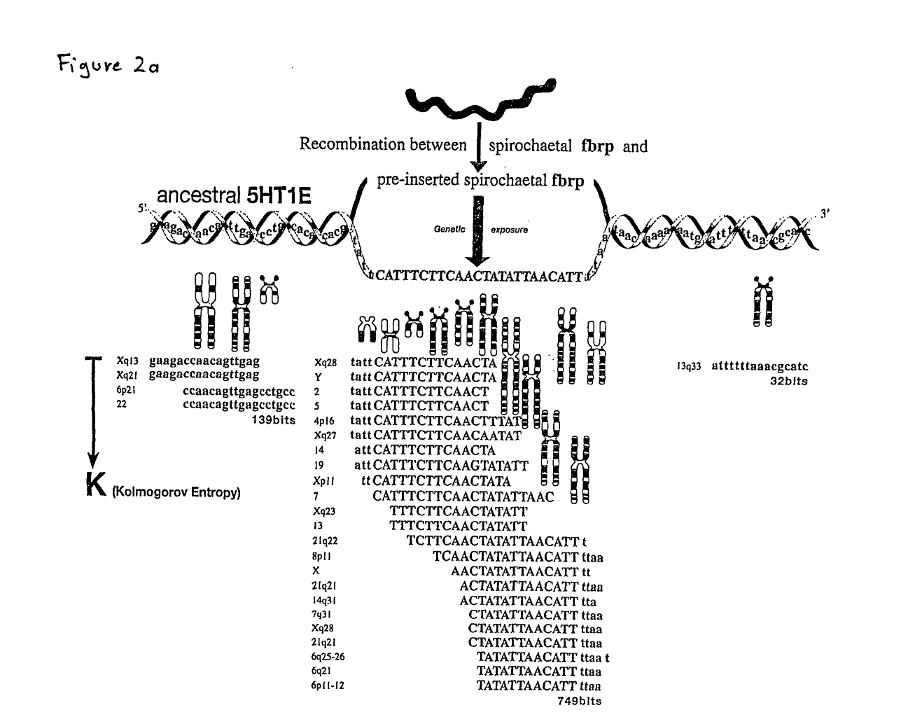

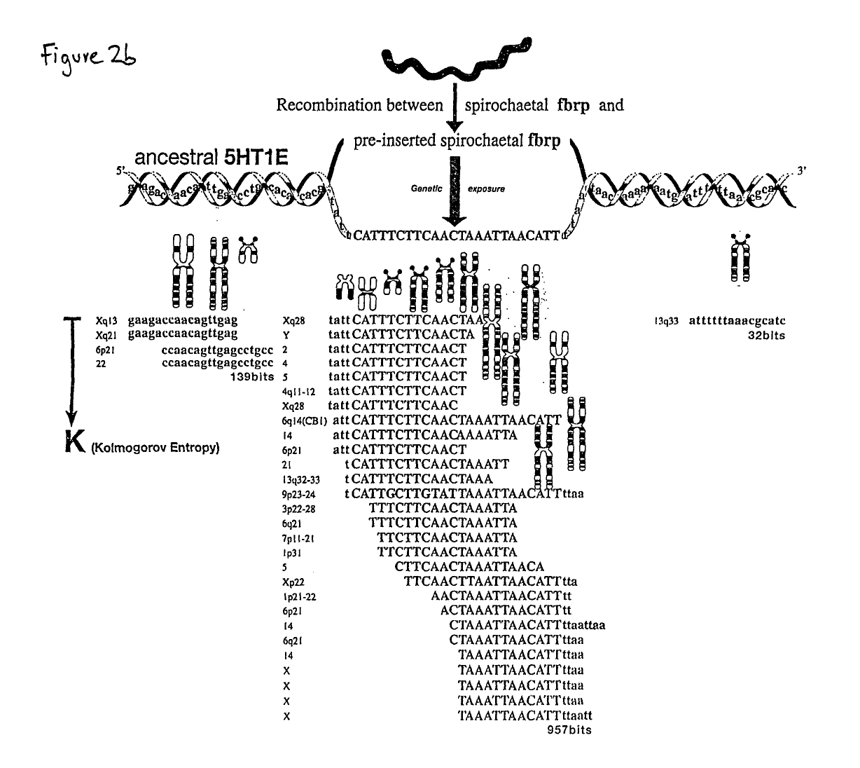

On the human CB1 gene, located within a candidate region for schizophrenia at 6q14 immediately adjacent to the 5HT1E (5-hydroxytryptamine) gene, a nucleotide sequence of (p115) originating from B. burgdorferi could be identified (Seq. Id. No. 1). Another ancestral spirochaetal inclusion on 5HT1E originates from a B. burgdorferi virulence factor, the flagellar basal-body rod protein (fbrp). It can still be found within the serotonin receptor 1E gene (5HT1E) on the mouse (Mus musculus) and rat (Rattus norvegicus). During phylogeny, multiple recombinations between the spirochaetal fbrp and its pre-inserted fbrp templates have exposed the identical sequences on the complementary strand on the double helix, including adjacent non-microbial nucleotides, to further recombination all over the human genome and a clustering of microbial virulence factors from Chlamydia muridarum iron binding protein, Plasmodium falciparum rhoptry and Staphylococcus aureus penicilllin binding protein, a specific gene conversion occured (see FIG. 1 and FIG. 2). Through infectious recombination, B. burgdorferi subsequently introduced p115 into our ancestral genome resulting in a genetic overlap with fbrp. Containing the poly-A signal (AATAAA), p115 thus introduced the code for the translation and actual genetic expression of CE1.

### Example 3

No Point Mutations Within the Polyadenylation Signal (AATAAA)

With the introduction and natural selection of the inserted polyadenylation site, lateral gene transfer have effectively influenced the genetic expression of CB1. Presenting save havens for microbial DNA there are no point mutations on this important signal, for a change in the signal would disrupt the genetic expression of CB1. Suppose the mutually advantageous sequences on the polyadenylation signals recombine again with slightly dissimilar spirochaetal strands, the subsequent mismatch repair mutations will be deleterious for both hosts and parasites. Being located on an important 3′ regulatory code, whose mutational rate runs slower than the molecular clock of silent point mutations in protein coding regions, microbial sequences are thus better protected from mutations and hence stabilised.

### Example 4

DNA of B. burgdorferi Underlies the Schizophrenic Genotype

That lateral gene transfer has influenced the evolution of higher eukaryotes, such as mammals is counter-current to established views. B. burgdorferi appears to be excluded from the benefits of the extensive lateral gene transfer between microorganisms, however, as an intracellular parasite with an incomplete genome B. burgdorferi has a direct access to host genes, which it exploits for replication, and not to be recognised as foreign, the spirochete depends on its own sequences within the human genome. Through molecular mimicry of fbpr antigens within the host's CD45 leukocyte defence system and a structure homologous to nucleoprotein, B. burgdorferi might, for example, dispose of a protective shield at the DNA and protein level, respectively.

Dissimilarity and mismatch mutations between B. burgdorferi and its pre-inserted human templates may nevertheless occur, resulting from genetically induced variability by virulence factor operons, osp A and B, or, alternatively, from a genomic decay in plasmids (Casjens et al., Mol. Microbiol. 2000, 35, 490-516) borrelia re-infection causing putative mutations in AATAAA. Intriguingly, by a single frame-shift mutation within the original translocase, the same process of genomic decay has apparently incapacitated B. burgdorferi to translocate further genetic templates into its hosts (Casjens et al., Mol. Microbiol. 2000, 35, 490-516). Whereas point-mutations within the poly-A signal would impair the genetic expression, a change in the adenine content of polyadenylation tail (Alberts et al., Molecular Biology of the Cell, 1994, Garland Publishing), would alternatively enhance or reduce the ribosomal translation of CB1. Several mutations at an early blastular stage can lead to different (chimeral) expressions of CB1 (and perhaps other genes) and account for the reported continuum of major psychoses between schizophrenia and bipolar manic-depression.

Dissimilarity and infectious recombination between protein coding sequences of the human chromosome associated protein hCAP and borrelia P115 (63% identities with P115) could, on the other hand, account for the altered leukocyte chromatin ultra-structure reported in schizophrenic patients.

Genetic epidemiology which has provided consistent evidence over many years that schizophrenia has a genetic component and that this genetic component is complex and polygenic (Riley and McGuffin, Am J Med Genet 2000, 97, 23-44) does not challenge the main tenet of the present disclosure. The many reported coincident regions for schizophrenia and the sequences of B. burgdorferi within the human genome just reflect two different aspects of the same phenomenon—infectious recombination (see FIG. 1 and FIG. 2). FIG. 1 shows that multiple recombinations between the spirochaetal fbrp and its pre-inserted fbrp template on ancestral 5HT1E exposed the complementary strand on the double helix, including adjacent non-microbial nucleotides, to further recombination with both microbial and ancestral DNA. This has led to a gene conversion from 5HT1E onto CB1, which are both located adjacent to each other on the 6q14 candidate region for schizophrenia. Since the first three nucleotides (att) can still be found on 5HT1E of the mouse (Mus musculus) and rat (Rattus norvegicus), but not on fbrp of Borrelia burgdorferi, the spirochaetal template on the CB1 gene originates from ancient 5HT1E already containing the spirochaetal inclusion, and not from a direct transposition of B. burgdorferi onto 6q14. Observe the ‘loophole’ mutation, inserting an additional adenine into CB1. Homologous recombination and mismatch-repair mutation between B. burgdorferi p115 and the borrelia template fbrp subsequently introduced the polyadenylation signal AATAAA, which now encodes the ribosomal translation and genetic expression of CB1.

FIGS. 2a and 2b show that infectious recombinations between B. burgdorferi fbrp and its pre-inserted fbrp template on ancestral 5HT1E repeatedly exposed the complementary strand on the double helix, including adjacent non-microbial nucleotides, to further recombinations with ancestral DNA. This has occurred before and after the point mutation from adenine to thymin A-T within the pre-inserted fbrp on 5HT1E. In comparison to the adjacent non-exposed control sequences of the same length (32 base pairs), genetic exposure has thus given rise to a high number of translocations within the human genome. Most translocations are, in addition to chromosome X, located on 6q reflecting their original spread from ancestral 5HT1E on nearby 6q14. Multiple translocations into 6q21, 6p21 and Xq28 also point to candidate regions of major psychoses, that correlate with the highest lod-score for schizophrenia at 6q21, the candidate region for schizophrenia at 6p22 and the candidate region for bipolar manic depression at Xq28.

The phylogenetic trace implies that such recombinations have occured over and over again, and that novel mutations within pre-inserted spirochaetal templates are likely to occur. The excess of winter-spring births in sporadic schizophrenia, which follows the spring-autumn season of tick born infection, and its geographical overlap of the tick vector with areas of increased risk for sporadic schizophrenia (Brown, Schizophr Bull 1994, 20, 755-75) suggests B. burgdorferi as the prime infectious candidate. Only a few other micro-organisms express nucleotide sequences for transplacental penetration, and fetal infection, which are homologous to those of their host and thereby not viewed as foreign. With only an incomplete genome at its disposal, B. burgdorferi exploits and therefore interferes with the genetic machinery of its host cell for replication (Fraser et al., Nature 1997, 390, 580-86). If in analogy to the neurotropic murine leukemia virus, B. burgdorferi infects oligo-cellular blastulas in early pregnancy, a horizontal transfection of germ line cells would lead to a vertical transmission of the disease. This, in fact, occurs during the transmission of B. burgdorferi from the adult ixoid tick into eggs and larvae, which can thus become infectious without prior blood meal, and sporadic prenatal B. burgdorferi infection have indeed been reported in human beings (Steere in Principles and Practice of Infectious Diseases. Philadelphia: Churchill Livingstone, 2000, 2504-2518). By adding genetically vulnerable cases to the population human germ-line transfection by B. burgdorferi would thus explain the continued presence of schizophrenia at high prevalence, despite the fact that the disease confers reduced procreational fitness and fertility.

The association between level of cannabis consumption and development of schizophrenia during a 15-year follow-up was studied in a cohort of 45,570 Swedish conscripts. The relative risk for schizophrenia among high consumers of cannabis (use on more than fifty occasions) was 6.0 (95% confidence interval 4.08.9) compared with non-users. Persistence of the association after allowance for other psychiatric illness and social background indicated that cannabis is an independent risk factor for schizophrenia (Andreasson et al., Lancet 1987 Dec. 26; 2 (8574:1483-6).

### Example 5

Significant CB1-D1 Protein Homology Versus CB1-D2 Nucleotide Homology

There are significant homologies, which cannot be reduced to lateral gene transfer. CB1 shows significant homology with the dopamine D1—a type I G-protein coupled receptor—at the amino-acid level (Score=71.0 bits (171), Expect=2e−11, Identities=75/320 (23%), Positives=137/320 (42%), Gaps=50/320 (15%) (Online Mendelian Inheritance in Man, OMIM 2000, Center for Medical Genetics, John Hopkins University (Baltimore, Md.) and National Center for Biotechnology Information, (Bethesda, National Library of Medicine, 2000). Other homologous proteins comprise G-protein coupled receptors that receive information from outside the cell, such as olfaction or vision. However, owing to one specific sequence of 19 homologous base pairs at the nucleotide level, it is the dopamine 2 receptor (D2)—a type II G-protein coupled receptor—which shows significant DNA homology to CB1 (Score=37.2 bits (19), Identities=19/19 (100%). The circumscribed homology between CB1 and the D2 receptor, which is an important pharmacological target for the treatment of schizophrenia, encodes the seventh transmembrane loop which is known for its inhibitory-mode (i-mode) of metabotropic action. Apart from a silent point mutation from GTG to GTC in the rat D2 receptor gene (Rattus norvegicus), which has occurred after the phylogenetic rat mouse divergence 35 million years ago, this homologous nucleotide sequence can be found an all sequenced primate (Macaca mulatta, Cercopithecus aethiops, H. sapiens; OMIM, 2000) and rodent (Mus musculus) D2 receptor genes.

An early prenatal event interfering with neuronal migration from inner to outer cortical laminae in mid pregnancy most likely underlies the consistent pattern of cellular disarray observed in schizophrenic brains. If the expression of higher levels of CB1 within outer cortical compared to inter-cortical layers (Glass et al., 1997, Neuroscience, 77, 299-318) resulted from a CB1 mediated migration of neurons, the inter-cortical disarray could be explained by a knock-out of CB1, whose metabotropic i-mode has recently been reported to be crucial for cellular migration (Song and Zhong, Journal of Pharmacology and Experimental Therapeutics, 2000, 294, 204-209). The distribution of CB1, furthermore, exactly mirrors the macro-anatomic regions mainly affected in schizophrenia (Schultz and Andreasen, 1999, Lancet, 353, 1425-30).

Through a dysinhibition of the i-mode, spatial memories (hippocampal long-term potentiation) of CB1 knock-out mice are enhanced (Bohme et al., 2000, Neuroscience, 95, 5-7) and goal directed, temporal memories decreased. This mnemonic effect, however, not only parallels the pattern of spatio-temporal distortions in schizophrenia. Despite the fact that patients with Alzheimer's dementia and tertiary neurosyphilis do hallucinate, they apparently do not remember, or reconnect their hallucinations with a fixed delusion. The difference between dementia praecox (schizophrenia) and syphilitic dementia is that without memory hallucinations are lost, and that the thoughts of schizophrenics are flooded with fixed hallucinations, expanding into overt delusions. Furthermore, CB1 knock-out mice do show reduced exploratory, goal-directed behaviours (Steiner et al. Proc. Natl. Acad. Sci. USA, 2000, 96, 5786-5790); symptoms that appear to be among the most robust indices in schizophrenia. Without time-bridging working memory, no creative speech and no logical thinking would be possible. A disruption of CB1, which reaches its highest levels in the left-hemispheric area of Wernicke, would thus account for the impairment of goal directed behaviour and speech—the highest form of sequential behaviour in man—being more stereotyped in schizophrenic patients than in healthy persons.

### Example 6

The phylogenetic traces of microbial insertions into the human genome were investigated and the genetic mechanism were analysed, by which bacterial virulence factors and mobile elements from intracellular parasites could disrupt candidate genes for schizophrenia and dementia. This was done by coincident DNA homology BLAST searches between neurotropic microorganisms, the central cannabinoid receptor CB1, and other known dementia genes, whose complete sequences with 3′UTR and polyadenylation signals are entered on Gene-Bank databases. Several such genes have now been characterised including those of ApoE4 Alzheimer Disease type II (AD2) and presenilin 2 (AD 4). Positional cloning and sequencing has also been carried out in other dementias including Familial British Dementia (with mutations in the gene for integral membrane protein 2A), Hereditary Multi-infarct Dementia (Notch-3 gene), primary X-linked mental retardation (oligophrenin 1 gene), Frontotemporal Lobe Dementia, Autosomal Dominant Parkinson Lewy-Body Dementia and Familial Parkinson disease type I (with mutations in the alpha synuclein gene). The genomic facilities used are accessible at Online Mendelian Inheritance of Man (OMIM) and very much recommended to anybody interested in applied medical genetics.

While there are shown and described presently preferred embodiments of the invention, it is to be distinctly understood that the invention is not limited thereto but may be otherwise variously embodied and practiced within the scope of the following claims.

TABLE 1A
SCHIZOPHRENIA

CB1
1st & 2nd borrelia integrations into ancestral 5HT-CB1 and introduction of poly-A signal

| | | | |
|---|---|---|---|
| | Borrelia (fbrp) | (357) CATTTCTTCAACTAAATTAACATT(334)5′ | 18 |
| 5HT1E/ | | AttCATTTCTTCAACTAAATTAACATT | 19 |
| CB1 | | AttCATTTCTTCAACTAAATTAACATT | |
| (anc. 1) | | | |
| 6q14 | Borrelia (P115) | (4133) GATTCAAATAAAAATTCTAAATTACCAT(4160)3′ | 1 |

Chlamydia integration into ancestral rodent CB1

| | | | |
|---|---|---|---|
| | Chlamydia | (8221) TTACCTGGACTCAAATAAAAGT(8242)3′ | 20 |
| CB1 | (ibp) | GAATCAAATAAAAATTCTAGATTACCATgaagaacata | 22 |
| (anc. 2) | | | |
| CB1 | | (5430) TTACCTGGAATCAAATAAAAGTTCTAGATTATCACg (5465)3′ | 21 |
| (rat) | | | |

Chlamydia muridarum integration into ancestral primate CB1

| | | | |
|---|---|---|---|
| | Chlamydia | (8219) AGTTACCTGGACTCAAATAAAA(8240)3′ | 2 |
| CB1 | (ibp) | GAATCAAATAAAAATTCTAGATTACCATgaagaacata | 22 |
| (anc. 2) | | | |
| CB1 | | (5484) AGTTACCTGGAATCAAATAAAAATTCTAGATTACCATgaagaacata(5530) | 47 |
| (human)/ | | | |
| 6q14 | | | |

3rd borrelia infection - predicted mutations by virulence factor or plasmid
of human CB1 poly-A signal

| | | |
|---|---|---|
| Borrelia burgdorferi (ospA) | (41) ATAATAATTCTAAATTA(25)5′ | 24 |
| Borrelia garinii (ospA) | (229) ATAATAATTCTAAATTA(213)5′ | 23 |
| Borrelia (plasmids;i.e.1p28-1) | (7553) AATATAAATTCTATAT (7568)3′ | 25 |

Microtubule associated protein 2C (MAP2C)
Borrelia burgdorferi inclusion from pseudouridylate synthetase

| | | | |
|---|---|---|---|
| MAP2C | | (3634) AAACTCAGAAAATAAAATGT3′(3653) | 54 |
| | Borrelia (pus) | (2645) AAACTCAGAAAATAAAATGT5′(2626) | 16 |
| | Borrelia (slipp) reinfection | (40665) CAGAAAATAAAAT 5′(40653) | 55 |

TABLE 1B

DEMENTIA
APOLIPOPROTEIN E (Apo E) Alzheimer's diease type 2 (AD2)
Borrelia burgdorferi surface protein integrated within ancestral ApoEe4

| | | | |
|---|---|---|---|
| | Borrelia (lipP) | (11) GTTTAATAAAAATT(24)3′ | 3 |
| Apo E (anc.) | | GTTTAATAAAAATT | 26 |

Chlamydia pneumoniae integration into ancestral ApoEe4

| | | | |
|---|---|---|---|
| | Chlamydia | (1063) AGTTTAATAAAGATT (1049)5′ | 4 |
| Apo E (anc.) | | GTTTAATAAAAATT | 50 |
| Apo E | | (4611) AGTTTAATAAAGATTca(4627)3′ | 27 |

borrelia infection - predicted mutations by virulence factors

| | | |
|---|---|---|
| Borrelia (acrB) | (8106) TTTAAAAAAGATTCA (8120)3′ | 28 |
| Borrelia (comp.loc) | (11560) TTTAAAAAAGATTCA (11546)5′ | 28 |

PRESENILIN 2 (PS2) - Alzheimer's disease type 4 (AD4)
Borrelia burgdorferi (sgp) stability governing protein integrated within presenilin 2
and possible predicted mutation by Borrelia reinfection

| | | | |
|---|---|---|---|
| | Borrelia (sgp) | (10599) ATACTAATATCAATAA (10614)3′ | 5 |
| PS 2 | | (1651) ATACTAATATCAATAAa(1667)3′ | 29 |
| | Borrelia (sgp) | (10599) ATACTAATATCAATAAT(10615)3′ | 30 |
| | reinfection | | |

AMYLOID PRECURSOR PROTEIN (APP) Alzheimer's disease type 1 (protease nexin-II)
No Borrelia but has Plasmodium falciparum inclusion and Salmonella typhimurium
transposable plasmid

| | | | |
|---|---|---|---|
| APP | | (3547) TTTTCATGTAAATAAATACATTCT(3570)3′ | 31 |
| | Salmonella plasmid traJ | (425) TGTAAATAAATACATTCT(442)3′ | 48 |
| | Plasmodium chromosome 3 | (79642) TTTTCATGTAAATAAATA (79625)5′ | 6 |
| | (hypothetical protein) | (141360) TTTCAGGTAAATAAATA (141344)5′ | 32 |

FAMILIAL BRITISH DEMENTIA (integral membrane protein 2B gene) Borrelia burgdorferi
linear plasmids lp25, lp36 and Plasmodium falciparum major merozoite surface
& receptor binding protein

| | | | | |
|---|---|---|---|---|
| FBD | | (1753) GATTTTTTCTTTAAATAAAAATAAGT(1778)3′ | 33 | |
| | Borrelia lp25 plasmid | (18048) TTTAAATAAAAATAAGT(18032)5′ | 7 | |
| | Borrelia lp 36 plasmid | (9312) TTTAAATAAAAATAAG (9327)3′ | 51 | |
| | Plasmodium (mmsp) | (1083) TTTTTTTTTTAAATAAAAATA (1103)3′ | 34 | |
| | Plasmodium (pfemp1) | (4682) | GATTTTTTCTTTAGATAAAAATAAG(4658)5′ | 8 |

Oligophrenin 1 (OPHN1)
Borr lia burgdorferi inclusion from tryptophanyl-t-RNA synthetase

| | | |
|---|---|---|
| OPHN 1 | (6648) CAAATAAAGTAGTAAAAGA(6666)3′ | 56 |
| Borrelia (trsa) | (101) CAAATAAAGTAGTAAAAGA(83)5′ | 17 |

HEREDITARY MULTI-INFARCT DEMENTIA (NOTCH 3 gene)
No Borrelia or plasmodium, but has Vibrio cholera virulenence factor
and Clostridium insertion

| | | | |
|---|---|---|---|
| DHMI | | (8048) CCTAATAAAGGAATAGTTAAC (8068)3′ | 35 |
| | Vibrio (ntno) | (4641) AATAAAGGAATAGTTAA (4657)3′ | 52 |
| | Clostridium (ctfA) | (2544) CCTAATAAAGGAATAG (2559)3′ | 53 |

FRONTOTEMPORAL LOBE DEMENTIA (gene for microtubule associated protein tau)
No Borrelia or plasmodium, but has Staphylococcus aureus antibiotic resistance plasmid

| | | | |
|---|---|---|---|
| MAP Tau | | (2276) GCTAGTAATAAAATAT(2291)3′ | 36 |
| | Staph. Plasmid pS194 | (2276) GCTAGTAATAAAATAT(2291)3′ | 36 |

PARKINSON DISEASE
AUTOSOMAL DOMINANT LEWY BODY (PDLBD) (alpha synuclein gene)
Borrelia burgdorferi inclusion from linear plasmid lp36

| | | | |
|---|---|---|---|
| PDLBD | | | |
| 2nd poly A | | (1521) ACAATAAATAATATTC(1536)3′ | 37 |
| signal | | | |
| | Borrelia lp36 | (13825) ACAATAAATAATATTC(13810)5′ | 9 |

PARKINSON DISEASE, FAMILIAL TYPE I (PD1) (alpha synuclein gene)
Borrelia burgdorferi linear plasmid 1p17, Plasmodium falciparum plasmid
and Escherichia coli pilus protein

| | | | |
|---|---|---|---|
| PD - 1 | | | |
| 3rd poly A signal | | (952) TAATAATAAAAATCATGCTT(971)3′ | 49 |
| | Borrelia lp17 plasmid | (9683) TAATAATAAAAATCAT (9668)5′ | 10 |
| | Plasmodium plasmid | (10913) AATAATAAAAATCATG (10928)3′ | 12 |
| | Escherichia (pilus protein) | (860) AATAATAAAAATCATGCTT(878)3′ | 11 |

TABLE 1C

MYOPATHY
MYOTONIC DYSTROPHY, 3′UTR & TRIPLET REPEAT
Borrelia burgdorferi and Chlamydia muridarum integration into
human myotonin protein kinase (Mt-PK)

| | | | |
|---|---|---|---|
| | Borrelia (fbrp) | (194) TCCGGAATAAAAGGCCCT(177)5′ | 13 |
| (Mt-PK, anc.) | | TCCGGAATAAAAGGCCCT | 38 |
| (Mt-PK) | | (2462) TCGCGAATAAAAGGCCCT(2479)3′ | 39 |
| | Chlamydia | (3551) GCGAATAAAAGGCCCT(3536)5′ | 14 |

TABLE 1 D
GLOBIN GENES - CONTROLS FOR MICROBIAL INSERTIONS

β-globin (chromosome 11)
β-globin chain versus β-thalassaemia

| | | |
|---|---|---|
| | Poly-A cleavage site (1736-1737) | |
| Normal β-globin | (1711) AATAAAAAACATTTATtttcattgca atgATGTATTTAAATta(1753) . . . | 40 |
| Rickettsia | (232700) AATAAAAAACATTTAT(232685)5′ | 15 |
| Borrelia (oppAIV) | (1143) ATGTATTTAAAT(1132)5′ | 41 |
| normal β-globin | (1754) tTTCTGAATATTTTACTAAAAA(1775)3′ | 42 |
| Borrelia (ospC) | (364) TTCTGAAGATTTTACTAAAAA(384)3′ | 43 |
| β-Thalassaemia | (1748) AATAAGAAACATTTATTTtcattgca(1773)3′ | 44 |
| Plasmodium falciparum | (470) ATAAGAAACATTTATTT (486)3′ | 45 |

α-globin (chromosome 16)

| | |
|---|---|
| α-globin chain (NM_000558) in lower case letters versus Plasmodiuni bergei (L21708) | |
| within 3′ α-globin in upper case letters | 46 |

| | | |
|---|---|---|
| α-1 and α-2 | 1 | actcttctgg tccccacaga ctcagagaga acccaccatg GTGCTGTCTC CTGCCGACAA |
| | 61 | GACCAACGTC AAGGCCGCCT GGGGTAAGGT CGGCGCGCAC GCTGGCGAGT ATGGTGCGGA |
| | 121 | GGCCCTGGAG AGGATGTTCC TGTCCTTCCC CACCACCAAG ACCTACTTCC CGCACTTCGA |
| | 181 | CCTGAGCCAC GGCTCTGCCC AGGTTAAGGG CCACGGCAAG AAGGTGGCCG ACGCCCTGAC |
| | 241 | CAACGCCGTG GCGCACGTGG ACGACATGCC CAACGCGCTG TCCGCCCTGA GCGACCTGCA |
| | 301 | CGCGCACAAG CTTCGGGTGG ACCCGGTCAA CTTCAAGCTC CTAAGCCACT GCCTGCTGGT |
| | 361 | GACCCTGGCC GCCCACCTCC CCGCCGAGTT CACCCCTGCG GTGCACGCCT CCCTGGACAA |
| | 421 | GTTCCTGGCT TCTGTGAGCA CCGTGCTGAC CTCCAAATAC CGTTAAGCtg gagcctcggt |
| | 481 | ggccatgctt cttgcccctt gggcctcccc ccagcccctc ctccccttcc tgcacccgta |
| | 541 | cccccgTGGT CTTTGAATAA AGTCTGAGTg ggcggc 3′ |

TABLE 1E
VIRULENCE FACTORS AND OTHER MICROBIAL INSERTIONS

Borrelia burgdorferi (p115): related to human chromosome associated protein responsible for DNA and intracellular
movement.
Borrelia b. (fbrp): flagellar basal rod protein for extracellular movement, chemotaxis, syringe mechanism for cell
injection.
Borrelia b. (ospA): outer surface protein A, antigen of genetically induced variation.
Borrelia b. (plasmid lp28-1) pseudogene on linear plasmid size 28, group 1, being the result of genomic decay.
Borrelia b. (lip P): lipoprotein P (homologous sequences on plasmids, i.e. lp38), anti-genetic surface protein.
Borrelia b. (acrB): acriflavine resistance protein.
Borrelia b. (comp.loc): competence locus with multiple homologous copies throughout genome.
Borrelia b. (sgp): stability governing protein for stabilisation of membrane.
Borrelia b. (oppAIV): oligopeptide permease; immediately adjacent to 3′poly-A cleavage site of (globin.
Borrelia b. (ospC): outer surface protein C; 18 base pair distant from 3′ poly-A cleavage site of (globin. Target of
Borrelia vaccine.
Borrelia b. (pus): pseudo uridylate synthase
Borrelia b. (slipp): surface lipoprotein p27
Borrelia b. (trsA): tryptophanyl t-RNA synthetase
Borrelia garini (ospA): outer surface protein A. Chlamydia muridarum (ibp) within CB1: iron binding protein to overcome
host barriers of low iron levels.
Chlamydia m. within Mt-Pk: phosphocarrier protein.
Chlamydia pneumonae: hypothetical protein.
Clostridium beijerinckii (ctfA): small subunit of coenzyme A transferase.
Escherichia coli pilus protein: responsible for cellular adherence and infection.
Plasmodium bergei within ( globin series: phosphoprotein mRNA.
Plasmodium falciparum within CB1: rhoptry associated protein (264)ATCAAATAAAAGTTCTA(280)3′ for erythrocyte
penetration.
Plasmodium f. within amyloid precursor protein: several sequences form chromosome 3 including hypothetical proteins.
Plasmodium f. (mmsp): major merozoite surface protein, expressed during sexual stage.
Plasmodium f. (pfemp1): Plasmodium falciparum-encoded protein on the surface of infected erythrocytes mediates receptor
binding.
Plasmodium f. in thalassaemia (globin: RNA polymerase III.
Rickettsia prowazekii in (globin: proline-betaine transporter for the reduction of osmotic stress in host environment.
Vibrio cholerae (ntno): Na+-translocating NADH-ubiquinone oxidoreductase enzyme complex involved in flagella rotation.
Microbial virulence factors within the 3′ genetic hotspot of human disease. Base pairs originating form the lateral
gene transfer of microbial nucleotides are indicated in upper case, and non-microbial nucleotides in lower case
letters. There are normally no point mutations on the polyadenylation signal, whose non-redundant code protects the
microbial inclusions from mutations. Homologous recombination between B. burgdorferi p115 and the borrelia template
fbrp originating from ancestral 5HT1E has introduced the polyadenylation signal AATAAA into CB1. Note that
recombinational mismatch-repair has inserted an additional adenine into CB1. A point mutation from A to G has through a
reduction of the adenine content led to a shortening of the 3′ polyadenylation tail of the rat. Within human CB1 and
rat CB1, almost identical, but independent insertions of C. muridarium nucleotides must have occurred twice. This
reoccurrence emphasises the attraction CB1 and its polyadenylation signal exerts on microbial DNA to recombine. In the
case of dissimilarity (i.e. AATATA) mismatch repair mutations might, in analogy to the genetic knock-out of globin in
thalassaemia, result in a knock out of CB1 and other neurological candidate genes.

1. Use of a DNA sequence comprising a fragment of a nucleic acid encoding a putative microbial virulence factor as means for the identification of a disease caused by mutations or a genetic predisposition thereof.

2. Use of claim 1 wherein said virulence factor is located on a linear or cirular chromosome or a plasmid.

3. Use of claim 1 or 2 wherein said virulence factor stems from a intracellular microorganism.

4. Use of claim 1 or 2 wherein said virulence factor stems from a non-intracellular pathogen and is part of a cluster shared by intracellular microorganisms.

5. Use of claim 1 wherein said microorganism is selected from the group consisting of Borrelia species, Chlamydia species, Escherichia sp., Plasmodium species and Rickettsia species.

6. Use of anyone of claims 1 to 5 wherein said fragment is selected from the group consisting of Seq. Id. No. 1 to Seq. Id. No. 17.

7. Use of anyone of claims 1 to 4 wherein said sequence comprises a mutation, either caused by by the same or a different species, preferably within the polyadenylation signal sequence.

8. Use of anyone of claims 1 to 7 wherein said disease is a human disease.

9. Use of claim 8 wherein said human disease is selected from the group consisting of schizophrenia, Alzheimer disease, Parkinson disease, Myopathy and other forms of dementias.

10. Use of claim 8 wherein said human disease constitutes a predisposition or a genetic variation, the pathological manifestation of which is triggered by medicaments or drugs.

11. Use of claim 10 wherein said drug is cannabis.

12. Use of claim 11 wherein said pathological manifestation comprises any form of dementia, schizophrenia, or related psychatric disorders.

13. A method for the identification of a disease or a genetic predisposition thereof, which comprises detecting the presence in a tissue-or blood sample of a subject a mutation within a nucleic acid sequence selected from the group consisting of Seq. Id. No.1 to Seq. Id. No. 17 and said sequence is part of a gene of said subject.

14. The method of claim 13 wherein said tissue sample is a foetal graft for neurotransplantation.

15. The method according to claim 13 or 14, wherein said sequence is inserted in the 3′UTR of said gene.

16. The method according to anyone of claims 13 to 15, wherein said mutation is found in the polyadenylation signal of said gene.

17. The method according to anyone of claims 13 to 16, wherein said mutation affects the expression of the protein encoded by said gene.

18. The method according to anyone of claims 13 to 17, wherein said gene is selected from the group consisting of Cannabinoid receptor 1 gene, MAP 2C gene, apolipoprotein E gene, presenilin 2 gene, integral membrane protein 2B gene, alpha synuclein gene, oligophrenin 1 gene and myotonin protein kinase gene.

19. A transgenic non human animal whose genome comprises a partially or completely inactivated endogenous gene as defined in claim 18, wherein said inactivation is due to at least one mutation in its 3′ untranslated region, said mutation leading to inhibition or suppression of the subsequent gene translation.

20. The transgenic non-human animal of claim 19, wherein the mutation is located in the nucleic acid sequence following the polyadenylation signal, more preferably in the polyadenylation sequence of said gene.

21. The transgenic non-human animal of claim 20, wherein said mutation is a point mutation.

22. The transgenic non-human animal according to anyone of claims 19 to 21, wherein said animal is a mammal, in particular a rodent.

23. The transgenic non-human animal of claim 22, wherein said animal is a mouse or a rat.

24. The transgenic non-human animal according to anyone of claims 19 to 23, wherein said inactivation is a homozygous or a heterozygous inactivation.

25. Use of a transgenic non-human animal according to anyone of claims 19 to 24 for the identification of compounds that have an effect on the activity, expression or regulation of the translated protein.

26. A method of screening compounds that have an effect on the activity, expression or regulation of a protein encoded by a gene according to claim 18 comprising introducing a compound in an animal according to anyone of claims 19 to 24 and monitoring behavioural changes in said animal as compared to a control animal.

27. Use of a transgenic non-human animal whose genome comprises a non-functional endogenous CB1 gene for the identification of compounds that have an effect on the activity, expression or regulation of CB1 protein.

28. A DNA and/or RNA chip comprising at least one of the nucleic acid sequences selected from the group consisting of Seq. Id. No. 1 to Seq. Id. No. 17.
